(19) United States
(12) Patent Application Publication
(10) Pub. No.: US 2026/0358224 A1
(43) Pub. Date: Nov. 12, 2026

(54) SYSTEM AND METHOD FOR MEASURING PARTICLES IN ULTRAPURE WATER

(71) Applicant: Nomura Micro Science Co., Ltd., Kanagawa (JP)

(72) Inventors: Hiroshi KIMOTO, Kanagawa (JP); Masamitsu IIYAMA, Kanagawa (JP); Yuya HIRAHARA, Kanagawa (JP)

(73) Assignee: Nomura Micro Science Co., Ltd., Kanagawa (JP)

(21) Appl. No.: 19/573,652

(22) Filed: Mar. 20, 2026

(30) Foreign Application Priority Data
May 7, 2025 (JP) 2025-077097

(51) Int. Cl. G01N 15/06; G01N 33/18

(52) U.S. Cl. CPC G01N 15/0618; G01N 33/18

(57) ABSTRACT

A particle measurement method includes: accumulating particles on an ultrafiltration membrane by filtering ultrapure water to be measured through an ultrafiltration membrane; and extruding the particles accumulated on the ultrafiltration membrane to the particle measurement apparatus together with concentrate water filtered through the ultrafiltration membrane.

### CROSS-REFERENCE TO RELATED APPLICATIONS

This application claims the benefit of priority under 35 U.S.C. § 119 to Japanese Patent Application No. 2025-077097, filed May 7, 2025, the content of which is incorporated herein by reference in its entirety.

### TECHNICAL FIELD

The present disclosure relates to a system and method for measuring particles in ultrapure water.

### DISCUSSION OF THE BACKGROUND

The description in this section merely provides information of background related to the present disclosure, and may not necessarily constitute the prior art.

Ultrapure water is used in the cleaning process in a semiconductor manufacturing step. Particles mixed into ultrapure water may cause defects in semiconductor devices, such defects seriously affecting the yield and performance of semiconductor devices. For this reason, techniques for removing particles from ultrapure water and for measuring the number of particles in ultrapure water are important.

For example, as a method for measuring the number of particles in ultrapure water, Japanese Patent Application Laid-Open No. 2008-241584 proposes a method including: separating ultrapure water containing particles into concentrate water with a high particle concentration and a permeate water with a low particle concentration using a hollow-fiber ultrafiltration membrane; then measuring the number of particles in the concentrate water; and calculating the number of particles contained in the ultrapure water based on a concentration factor and the measured number of particles. For the purposes such as detecting particles in a liquid, Japanese Patent Application Laid-Open No. 2025-032796 proposes a technique with which particles are first accumulated on a filtration membrane by performing dead-end filtering of a liquid and then recovered by backwashing. In this technique, particles in the liquid are accumulated on the membrane surface by dead-end filtration at high flow rates using a large-area ultrafiltration membrane, the filtration membrane is backwashed with alkaline functional water to remove the accumulated particles from the membrane surface, and then the accumulated particles are recovered by a filtration membrane for capturing particles.

### SUMMARY

A particle measurement method using a particle measurement apparatus, the method including an accumulating process of filtering ultrapure water to be measured through the ultrafiltration membrane to accumulate particles on the ultrafiltration membrane, and an extruding process of forcing the particles accumulated on the ultrafiltration membrane, together with concentrate water by the ultrafiltration membrane, to the particle measurement apparatus.

### Technical Problem

It has been found that in the method of concentrating particles using the ultrafiltration membranes, particles adhere to or accumulate on the surface or in filtration pores of the ultrafiltration membrane, and therefore the number of particles (particle concentration) in the ultrapure water, which has been calculated based on the concentration factor and the measured number of particles, does not reflect the actual number of particles. In particular, as the number of particles in ultrapure water becomes extremely small, the influence on the measured concentration becomes non-negligible even if the amount of particles adhering to the ultrafiltration membrane is small. Further, in the technique disclosed in Japanese Patent Application Laid-Open No. 2025-032796, in which a liquid is entirely filtered through a filtration membrane to accumulate particles on the membrane and the particles are then recovered by backwashing, the entire filtration is performed at a high flow rate. As a result, particles in the liquid tend to penetrate into the interior of pores of the membrane, giving rise to a concern that recovery by backwashing may be insufficient. In addition, the alkaline functional water used in backwashing may cause aggregation of particles, which may make analysis difficult. Moreover, since alkaline functional water can cause malfunction or damage of a particle counter in the above-described techniques, the captured particles cannot be measured using a particle counter.

### Solution to Problem

The present disclosure has been made to solve the above-described problems, and an object thereof is to provide a particle measurement method and a particle measurement system that are capable of measuring a particle concentration in ultrapure water with high accuracy.

The particle measurement method and particle measurement system according to an embodiment have the following configurations.
[1] A particle measurement method using a particle measurement apparatus, the method including:
   an accumulation process of filtering ultrapure water to be measured through an ultrafiltration membrane to accumulate particles on the ultrafiltration membrane; and
   an extrusion process of forcing the particles accumulated on the ultrafiltration membrane, together with concentrate water by the ultrafiltration membrane, to the particle measurement apparatus.
[2] The particle measurement method according to the aspect [1], wherein, in the accumulation process, dead-end filtration is performed.
[3] The particle measurement method according to the aspect [1], wherein the number of particles on the ultrafiltration membrane after the accumulation process is 4×10⁶ particles/cm² or more and 5×10¹⁰ particles/cm² or less.
[4] The particle measurement method according to the aspect [1], wherein a total amount of concentrate water flowing out of the ultrafiltration membrane in the extrusion process is 0.05 times or more and 0.2 times or less a total amount of the ultrapure water supplied to the ultrafiltration membrane in the accumulation process
[5] The particle measurement method according to the aspect [1], wherein continuous measurement is performed by alternately performing the accumulation process and the extrusion process.
[6] The particle measurement method according to the aspect [1], wherein an entire amount of the ultrapure water supplied to the ultrafiltration membrane in the extrusion process is allowed to flow out as the concentrate water.
[7] The particle measurement method according to the aspect [1], wherein a flow rate of the concentrate water by the ultrafiltration membrane in the extrusion process is 90% or more of a supply flow rate of the ultrapure water to the ultrafiltration membrane.
[8] The particle measurement method according to the aspect [1], wherein the number of particles with a particle diameter of 50 nm or more in the ultrapure water is 1 particle/L or more and 1000 particles/L or less.
[9] A particle measurement system, including:
   an ultrafiltration apparatus;
   a particle measurement apparatus connected to a concentrate side of the ultrafiltration apparatus;
   an accumulation mechanism configured to filter ultrapure water by supplying the ultrapure water to the ultrafiltration apparatus to accumulate particles in the ultrafiltration apparatus; and
   an extrusion mechanism configured to force the particles accumulated in the ultrafiltration apparatus to the particle measurement apparatus together with concentrate water in the ultrafiltration apparatus.
[10] The particle measurement system according to the aspect [9], including: a first valve provided on the concentrate side of the ultrafiltration apparatus; a second valve provided on a permeate side of the ultrafiltration apparatus; and a control apparatus configured to automatically control the first valve and the second valve, wherein the control apparatus constitutes the accumulation mechanism by reducing an opening degree of the first valve and opening the second valve, and the control apparatus constitutes the extrusion mechanism by opening the first valve and the second valve and reducing the opening degree of the second valve so that a flow rate at the first valve is 90% or more of a supply flow rate to the ultrafiltration apparatus.
[11] The particle measurement system according to the aspect [9], wherein the control apparatus constitutes the accumulation mechanism by closing the first valve and opening the second valve, and constitutes the extrusion mechanism by opening the first valve and closing the second valve.
[12] The particle measurement system according to the aspect [9], wherein the number of particles accumulated on a membrane surface of the ultrafiltration membrane by the accumulation mechanism is 4×10⁶/cm² or more and 5×10¹⁰/cm² or less.
[13] The particle measurement system according to the aspect [9], wherein the particle measurement apparatus measures particles online.
[14] The particle measurement system according to the aspect [9], wherein the accumulation mechanism and the extrusion mechanism are alternately operated.

Note that, if any, a sign “- (to)” may indicate a numerical range that includes numerical values before and after the sign.

### Advantageous Effects

According to the particle measurement method and a particle measurement system of embodiments, the concentration of particles in ultrapure water can be measured with high accuracy.

### BRIEF DESCRIPTION OF THE DRAWINGS

A more complete appreciation of the present disclosure and many of the attendant advantages thereof will be readily obtained as the same becomes better understood by reference to the following detailed description when considered in connection with the accompanying drawings, wherein:

FIG. 1 is a schematic block diagram illustrating a particle measurement system of an embodiment;

FIG. 2 is a schematic graph showing results of measurement of particles measured by a particle counter using the particle measurement system of the embodiment and the conventional technique;

FIG. 3 is a schematic block diagram illustrating a particle measurement system of another embodiment;

FIG. 4 is a schematic block diagram illustrating a particle measurement system of still another embodiment; and

FIG. 5 is a schematic block diagram illustrating a system for producing ultrapure water in an embodiment.

### DETAILED DESCRIPTION OF EMBODIMENTS

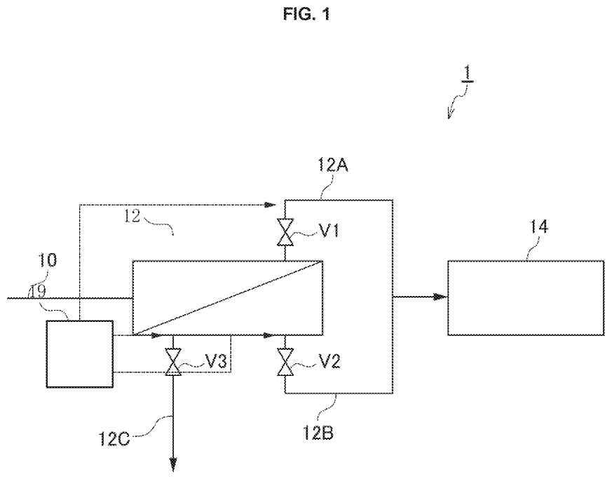

The particle measurement method and the particle measurement system of embodiments will be described below. FIG. 1 is a schematic block diagram illustrating a particle measurement system 1 of an embodiment. The particle measurement system 1 of the embodiment counts the number of particles in ultrapure water with the ultrapure water as an object to be measured, and measures the concentration of particles (particle concentration) per unit volume in sample water. The particle measurement system 1 can measure the concentration of particles in ultrapure water online or offline, and preferably measures the concentration online.

The particle measurement system 1 includes an ultrafiltration apparatus 12 and a particle measurement apparatus 14 in this order. The ultrafiltration apparatus 12 has an inlet connected to a supply pipe 10 through which ultrapure water is supplied to the ultrafiltration apparatus 12. The ultrafiltration apparatus 12 also has a concentrate water outlet that is connected to a discharge pipe 12A. The discharge pipe 12A is provided with a valve V1. The ultrafiltration apparatus 12 also has a permeate water outlet that is connected to a discharge pipe 12B and a discharge pipe 12C. The discharge pipe 12B is provided with a valve V2, and the discharge pipe 12C is provided with a valve V3. The valve V1, the valve V2, and the valve V3 are each an on-off valve or a flow control valve, and these valves may be automatically controlled by a control apparatus 19. As each valve, a multi-way valve may also be adopted. By using a multi-way valve, the functions of a plurality of valves provided in close proximity can be achieved by a single valve.

As the ultrafiltration apparatus 12, an ultrafiltration apparatus including an ultrafiltration membrane (UF) may be used. The ultrafiltration apparatus 12 is constituted of one or more ultrafiltration membrane modules. The ultrafiltration membrane module is configured, for example, to include an ultrafiltration membrane and a flow channel member for allowing water to be treated to flow through the ultrafiltration membrane, which are accommodated in a casing. As the ultrafiltration membrane module, a cartridge-type filtration membrane module can be used. The ultrafiltration membrane included in the ultrafiltration membrane module preferably has the form of hollow fiber membranes, but the ultrafiltration membrane may alternatively have the shape of spiral membranes, tubular membranes, flat membranes, or the like. The ultrafiltration membrane may be made of any material suitably used in the ultrafiltration membrane, such as polyethersulfone (PES), modified polyethersulfone (MPES), a fluororesin, polysulfone, polyvinylidene fluoride, polyethylene, and polypropylene. Among these, the material of the ultrafiltration membrane is preferably modified polyethersulfone. Since modified polyethersulfone has high hydrophilicity, particles adsorbed on a membrane surface of the ultrafiltration membrane are easily detached. Accordingly, particles accumulated on the ultrafiltration membrane by filtering ultrapure water through the ultrafiltration membrane in an accumulation process can be easily discharged from the ultrafiltration membrane when particles are extruded along with the concentrate water filtered through the ultrafiltration membrane to a particle measurement device, in a subsequent extrusion process, making modified polyethersulfone particularly suitable. The details will be described later. The ultrafiltration membrane used in the ultrafiltration apparatus 12 has a pore diameter large sufficient to capture particles, and preferably has a molecular weight cut-off in a range of 1 kDa or more and 100 kDa or less.

The particle measurement apparatus 14 is, for example, a light scattering-type particle counter that uses a laser as a light source. The particle measurement apparatus 14 can count the number of particles in sample water and as a result, can output the number of particles in water per unit volume as a measurement value of particle concentration. The particles measured by the particle measurement apparatus 14 have a particle diameter of, for example, 50 nm or more or 20 nm or more. As the light scattering-type particle counter, commercially available products, such as Ultra DI50 and Ultra DI20 manufactured by PMS, can be used. As the particle measurement apparatus 14 (particle counter), an acoustic-type particle counter may also be used, in addition to the light scattering-type particle counter. An acoustic-type particle counter emits acoustic waves having energy specific to particles to be detected into water to be treated. Pressures from cavitation events induced by particles that have received the acoustic waves are detected by a piezoelectric element of the acoustic-type particle counter. Cavitation pressures detected at or above a predetermined threshold are counted as particles. As the acoustic-type particle counter, a commercially available product may be used, for example, nanoPULS manufactured by OVIVO.

The measurement type of the particle counter is not limited to the light scattering type or the acoustic type, and any particle counter that can measure the particles online can be used.

A description will now be given of a particle measurement method using the particle measurement system 1. First, while ultrapure water to be measured is being supplied to the supply pipe 10, a control apparatus 19 controls the valves V1, V2, and V3 to reduce the opening degree of the valve V1 (first valve) and to open the valves V2 and V3 (which are collectively referred to as second valves). As a result, the accumulation mechanism is constituted, and particles in the ultrapure water supplied from the supply pipe 10 to the ultrafiltration apparatus 12 are captured by the ultrafiltration membrane of the ultrafiltration apparatus 12 and accumulated in the ultrafiltration apparatus 12 (accumulation process). The control apparatus 19 may be, for example, a processor or CPU.

The permeate water in the ultrafiltration apparatus 12 flows from the valve V2 through the discharge pipe 12B to the particle measurement apparatus 14. During this time, by adjusting the respective opening degrees of the valves V2 and V3, the amount of permeate water flowing through the valve V2 and the discharge pipe 12B to the particle measurement apparatus 14 can be adjusted. In particular, when the flow rate of permeate water flowing out through the discharge pipe 12B is increased, particles are pressed against the membrane surface of the ultrafiltration apparatus 12 and adhere to or accumulate on the membrane surface, thereby making it difficult for the particles to be discharged in the subsequent extrusion process. For this reason, in the accumulation process, the flow rate of permeable water, expressed as a flux value of the permeate water, is preferably less than 90 mm/min. In the accumulation process, the flow rate of permeate water, expressed as a flux value of the permeate water, is, for example, 10 mm/min or more and preferably 20 mm/min or more. When the flow rate falls within this range, 70% or more, preferably more than 75%, and more preferably 80% or more, of the number of particles flowing into the ultrafiltration apparatus 12 can be forced in the subsequent extrusion process.

In the accumulation process, the flow rate of concentrate water in the ultrafiltration apparatus 12 can be set to any flow rate at which particles can be accumulated in the ultrafiltration apparatus 12. Specifically, the flow rate of concentrate water in the ultrafiltration apparatus 12 is preferably 3% or less and more preferably 1% or less of the supply flow rate, and it is even more preferable that dead-end filtration that does not discharge concentrate water is performed in the accumulation process. In the accumulation process, dead-end filtration enables accurate measurement of the number of particles. However, when the flow rate of concentrate water falls within the above-described preferred range, the number of particles can be measured with accuracy equivalent to that achieved by dead-end filtration. Note that the flow rate is expressed as the volume (capacity) of water flowing per unit time unless otherwise noted. When the dead-end filtration that does not discharge concentrate water is performed in the accumulation process, the control apparatus 19 controls the valves V1, V2, and V3 to close the valve V1 and to open the valves V2 and V3 in the accumulation process.

Here, the permeate water in the ultrafiltration apparatus 12 in the accumulation process is discharged to the outside of the ultrafiltration apparatus 12 through the discharge pipe 12B and the discharge pipe 12C. The permeate water flowing out from the discharge pipe 12B is sent to the particle measurement apparatus 14. This configuration makes it possible to supply ultrapure water to the particle measurement apparatus 14 at all times and thereby maintain the apparatus in the operating state. The amount of ultrapure water allowed to flow to the particle measurement apparatus 14 may be any amount as long as the amount is sufficient to stably maintain the particle measurement apparatus 14 in the operating state.

After the accumulation process is performed for a predetermined period of time, the extrusion process is performed. While the ultrapure water to be measured is being supplied to the supply pipe 10, the control apparatus 19 controls the valves V1, V2, and V3 to open the valve V1, to reduce the opening degree of the valve V2, and to close the valve V3. As a result, the accumulation mechanism is constituted, and the ultrapure water supplied to the ultrafiltration apparatus 12 flows through the concentrate side of the ultrafiltration apparatus 12 to the particle measurement apparatus 14. Then, the particles accumulated in the ultrafiltration apparatus 12 are extruded by the ultrapure water and measured in the particle measurement apparatus 14. Here, in the extrusion process, the opening degree of the valve V2 is preferably reduced so as to allow the permeate water to flow to the particle measurement apparatus 14. In this case, the amount of permeate water allowed to flow to the particle measurement apparatus 14 may be any amount as long as the amount is sufficient to stably maintain particle measurement apparatus 14 in the operating state. In the particle measurement apparatus 14 of the present embodiment, the extrusion mechanism preferably reduces the opening degree of the valve V2 and closes the valve V3 so that the flow rate of concentrate water accounts for at least 90% of the supply flow rate of the ultrafiltration apparatus 12, and more preferably closes the valves V2 and V3 on the side of permeable water so that the total supply flow rate of the ultrafiltration apparatus 12 is equal to the flow rate of concentrate water. Thus, by increasing the flow rate of concentrate water, the entire amount of particles on the membrane surface of the ultrafiltration membrane can be extruded to the measuring side. Note that when the valve V3 is closed at the start of the extrusion process, the flow rate on the valve V1 side may temporarily increase. In such a case, a flow rate supplied to the particle counter fluctuates, thereby affecting measurement of the number of particles. To suppress effects of such flow rate fluctuations on the particle counter, a discharge line may be provided immediately upstream of the particle counter. By discharging a portion of the concentrate water flowing toward the particle counter through the discharge line, a supply flow rate to the particle counter can be maintained substantially constantly. In this case, it is possible to accurately measure the number of particles by monitoring the amount of discharge from the discharge line and correcting the measurement result of the particle counter based on the amount of discharge.

The total amount of concentrate water flowing out of the ultrafiltration membrane during the extrusion process is preferably 0.05 times or more and 0.2 times or less, and more preferably 0.08 times or more and 0.15 times or less, the total amount of ultrapure water supplied to the ultrafiltration membrane in the accumulation process. Satisfying these conditions makes it possible to sufficiently force the particles accumulated on the membrane surface, thereby facilitating acquisition of accurate measurement results of the number of particles.

The concentration of particles in ultrapure water, which is a measurement target of the particle measurement method of the present embodiment, is preferably 1 particle/L or more and 1000 particles/L or less. When the particles are aggregated in water or deposited on the membrane surface and the particles thereby bind together to form agglomerates (so-called “lumps”) of particles, they are not detached from the membrane surface after being adsorbed or captured on the membrane surface. On the other hand, when particles are adsorbed or captured on the membrane surface in a highly dispersed state, they can be easily detached from the membrane surface. In addition, when particles are maintained in a highly dispersed state without forming “lumps”, the particle measurement apparatus 14 can readily and accurately measure the number of particles and particle size thereof (particle diameter). When the concentration of particles in ultrapure water is 1000 particles/L, the highly dispersed state of particles is easily maintained, and in this highly dispersed state, the particles are accumulated in the ultrafiltration apparatus 12 during the accumulation process. The number of particles in ultrapure water is typically 10 particles/L or more and 100 particles/L or less. In the particle measurement method of the present embodiment, when the number of particles accumulated on the membrane surface of the ultrafiltration apparatus 12 reaches a certain number during the accumulation process, the particles deposited on the membrane surface become excessive and form “lumps” as described above. This prevents smooth outflow during the extrusion process. In the accumulation process, the number of particles on the membrane surface is, for example, 4×10⁶/cm²-5×10¹⁰/cm², and preferably 4×10¹⁰/cm² or less. With the above-mentioned number of particles, it is possible to prevent the particles from bonding together and to maintain easy detachment of the particles from the membrane.

Thus, according to the particle measurement method of the present embodiment, particles accumulated in the ultrafiltration apparatus 12 during the period of the accumulation process are sent to the particle measurement apparatus 14 in the extrusion process. Accordingly, the particles in the ultrapure water that have passed through the ultrafiltration apparatus 12 during the accumulation process are sent to the particle measurement apparatus 14 in a concentrated state. Therefore, all the particles accumulated in the ultrafiltration apparatus 12 can be sent to the particle measurement apparatus 14 and measured by the particle measurement apparatus 14 in the extrusion process. As a result, low-concentration particles can be measured with high accuracy. In addition, when the accumulation process and the extrusion process are alternately performed by alternately operating the accumulation mechanism and the extrusion mechanism by the control apparatus 19 or the like, continuous measurement of particles becomes possible.

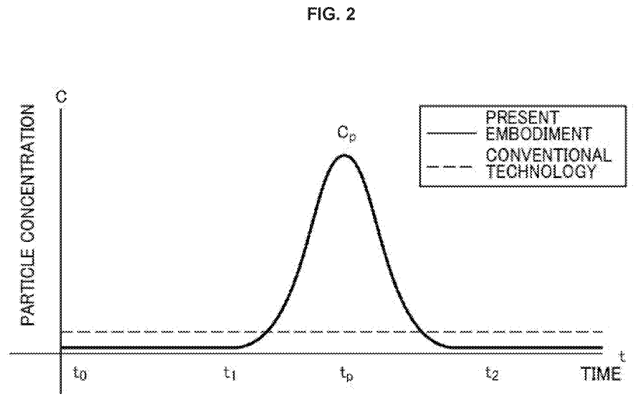

FIG. 2 schematically shows examples of the results of particle measurement with the particle measurement apparatus 14 in the particle measurement method of the embodiment and the conventional particle measurement method. A vertical axis represents a particle concentration measured by the particle measurement apparatus 14, and a horizontal axis represents time. In addition, a solid line indicates a change in concentration of particles measured by the particle measurement method of the present embodiment. A broken line shows a change in concentration by an analytical method in the conventional technology, with which the concentration of particles is analyzed by passing ultrapure water directly through the particle measurement apparatus without an ultrafiltration apparatus. In FIG. 2, t₀ denotes a start time of the accumulation process, t₁ denotes a start time of the extrusion process (that is, an end time of the accumulation process), and t₂ denotes an end time of the extrusion process. Here, in FIG. 2, a time required for water to reach the particle measurement apparatus 14 from the ultrafiltration apparatus 12 is assumed to be 0 (zero), that is, water discharged from the ultrafiltration apparatus 12 is assumed to reach the particle measurement apparatus 14 immediately after discharge.

In FIG. 2, an area of a region bounded by the graph between t₁ and t₂ and C(t) (particle concentration)=0, T=t₁, and T=t₂ represents the number of particles accumulated in the ultrafiltration apparatus 12 during the accumulation process.

From these results, the concentration of particles in ultrapure water can be expressed by the following Expression (1).

$$\begin{matrix}[ Expression 1 ] & \\ \frac{{\int }_{{t}_{1}}^{{t}_{2}} C ( t ) R Δ t}{( {t}_{1} - {t}_{0} ) \times {R}_{0}} & ( 1 )\end{matrix}$$

(in Expression (1), Δt denotes a differential of time t, C(t) denotes a measured value (concentration) obtained by the particle measurement apparatus 14 at time t, R denotes a supply flow rate to the particle measurement apparatus 14 during the extrusion process, and R₀ denotes a supply flow rate to the ultrafiltration apparatus 12 during the accumulation process).

According to the particle measurement method of the present embodiment, the concentration gradually increases from time t₁, reaches the peak concentration Cₚ at tₚ, and then decreases toward time t₂. Thus, the peak of the number of particles measured in the extrusion process is clearly observed. On the other hand, in the analytical apparatus in the conventional technology, the peak of concentration is not observed. This indicates that when the particles accumulated in the ultrafiltration apparatus 12 due to the accumulation process in the embodiment are supplied to the particle measurement apparatus 14 in the extrusion process, a peak appears due to transient high concentration, whereas in a conventional constant cross flow filtration, a change in concentration of concentrate water is small.

When particle concentration data cannot be obtained continuously or intermittently over time and only one or a few data points can be obtained at a single timing, sample water containing standard particles or the like may be prepared in advance, and the concentration of particles of the sample water may be measured beforehand. Then, based on the concentration of particles in the sample water and a peak value obtained from a measurement target, a particle concentration can be calculated using a calibration curve. Alternatively, a peak may be directly measured by adjusting operation such that a measurement timing of the particle measurement apparatus 14 matches a timing at which particles reach the particle measurement apparatus 14. In these cases, it is preferable that a supply flow rate of concentrate water to the particle measurement apparatus 14 be kept constant at all times, because variation in the supply flow rate of concentrate water causes a peak arrival time to fluctuate, thereby making it difficult to accurately determine a concentration.

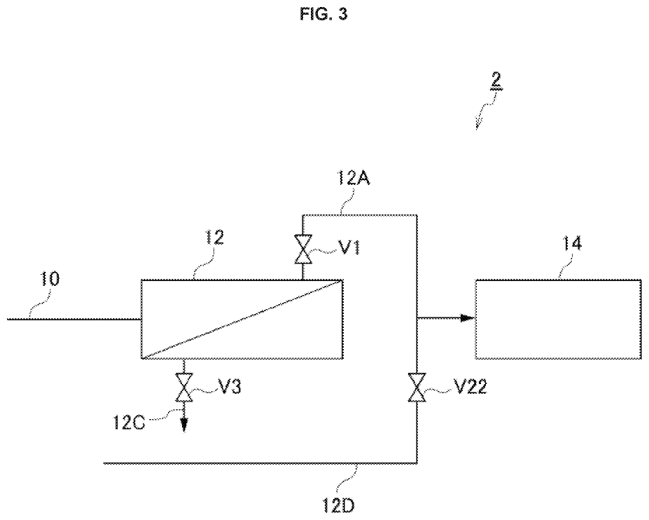

FIG. 3 illustrates a particle measurement system 2, in which ultrapure water is allowed to flow to the particle measurement apparatus 14 in the accumulation process. The particle measurement system 2 is different from the particle measurement system 1 illustrated in FIG. 1 in that the discharge pipe 12B of the particle measurement system 1 is replaced with an ultrapure water pipe 12D that allows ultrapure water to flow to the particle measurement apparatus 14. Since other components of the particle measurement system 2 are similar to those of the particle measurement system 1, the components demonstrating similar functions are designated by identical reference signs to omit detailed description thereof. In the particle measurement system 2, the ultrafiltration apparatus 12 has a concentrate water outlet that is connected to the discharge pipe 12A, and a permeate water outlet that is connected to the discharge pipe 12C. The ultrapure water pipe 12D is provided with a valve V22. The valve V22, similar to the valve V1, is an on-off valve or a flow control valve, and these valves may be automatically controlled by the control apparatus.

In the particle measurement system 2 illustrated in FIG. 3, in the accumulation process, while the ultrapure water to be measured is being supplied to the supply pipe 10, the control apparatus not illustrated controls the valves V1, V22, and V3 to close the valve V1 and to open the valves V22 and V3. As a result, dead-end filtration of the supply water is performed by the ultrafiltration apparatus 12, so that the entire amount of particles in the supply water is accumulated in the ultrafiltration apparatus 12.

Then, after the accumulation process is performed for a predetermined period of time, the extrusion process is performed. While the ultrapure water to be measured is being supplied to the supply pipe 10, the control apparatus not illustrated controls the valves V1, V22, and V3 to open the valve V1 and to close the valves V22 and V3. As a result, the entire amount of the ultrapure water supplied to the ultrafiltration apparatus 12 flows through the concentrate side of the ultrafiltration apparatus 12 to the particle measurement apparatus 14. With this process, the particles accumulated in the ultrafiltration apparatus 12 are extruded by the ultrapure water and measured in the particle measurement apparatus 14.

In addition, through the accumulation process and the extrusion process, ultrapure water flows through the ultrapure water pipe 12D to the particle measurement apparatus 14. This configuration makes it possible to maintain the particle measurement apparatus 14 in the operating state. The amount of ultrapure water allowed to flow to the particle measurement apparatus 14 may be the amount sufficient to stably maintain the particle measurement apparatus 14 in the operating state.

According to the particle measurement system 2 illustrated in FIG. 3, in the accumulation process, an entire amount of particles in ultrapure water supplied to the ultrafiltration apparatus 12 is accumulated in the ultrafiltration apparatus 12, thereby enabling more accurate measurement of particles.

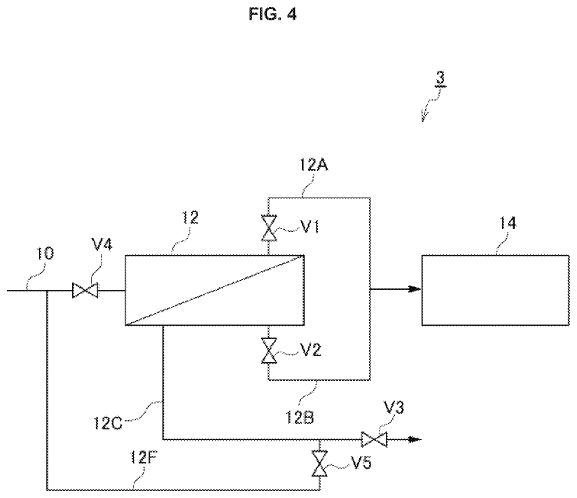

FIG. 4 illustrates a particle measurement system 3, in which ultrapure water is supplied to the ultrafiltration apparatus 12 from the permeate side to force particles in the extrusion process. In the particle measurement system 3 illustrated in FIG. 4, one end of a return pipe 12F is connected between the permeate water outlet of the ultrafiltration apparatus 12 and the valve V3 in the discharge pipe 12C. The other end of the return pipe 12F is connected to the supply pipe 10, and a valve V4 is provided on the downstream side of a connection point of the supply pipe 10 with the return pipe 12F. In addition, a valve V5 is also provided in the vicinity of a connection of the return pipe 12F with the discharge pipe 12C. The valves V4 and V5, similar to the valve V1 or the like, are each an on-off valve or a flow control valve, and these valves may be automatically controlled by the control apparatus.

In the particle measurement system 3 illustrated in FIG. 4, while the ultrapure water to be measured is being supplied to the supply pipe 10 in the accumulation process, the control apparatus not illustrated controls the valves V1 to V5 to close the valve V1 and valve V5 and to open the valves V2, V3, and V4. As a result, the ultrapure water is filtered by the ultrafiltration apparatus 12, so that the particles in the ultrapure water are accumulated in the ultrafiltration apparatus 12. The preferable aspect regarding the filtration condition in the accumulation process is similar to that of the particle measurement system 1 illustrated in FIG. 1.

Then, after the accumulation process is performed for a predetermined period of time, the extrusion process is performed. While the ultrapure water to be measured is being supplied to the supply pipe 10, the control apparatus not illustrated controls the valves V1 to V5 to open the valve V1 and valve V5 and to close the valves V2, V3, and V4. As a result, the entire amount of ultrapure water supplied to the supply pipe 10 flows through the return pipe 12F and flows back through the discharge pipe 12C, and flows to the particle measurement apparatus 14 through the concentrate side of the ultrafiltration apparatus 12. With this process, the particles accumulated in the ultrafiltration apparatus 12 are extruded by the ultrapure water from the concentrate side and measured in the particle measurement apparatus 14. Thus, since particles are easily detached from the membrane (loosely adhere to the membrane), the entire amount of particles on the membrane surface of the ultrafiltration membrane can be extruded to the measuring side. Therefore, the particles can be extruded without using hydrogen water or functional water containing chemicals.

According to the particle measurement system 3 illustrated in FIG. 4, in the extrusion process, particles are allowed to flow to the particle measurement apparatus 14 by supplying ultrapure water from the permeate side of the ultrafiltration apparatus 12 to the concentrate side to perform backwashing. This allows accumulated particles to more accurately flow to the particle measurement apparatus 14, so that further highly accurate measurement of the particles are possible.

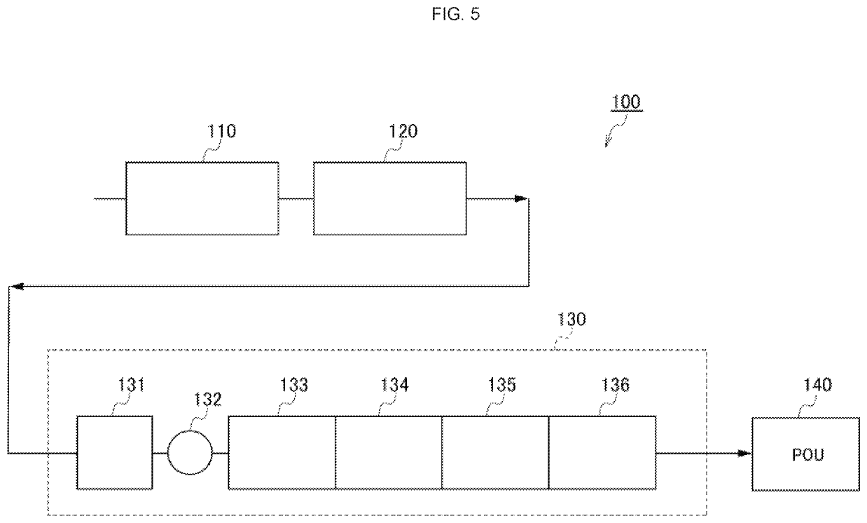

The ultrapure water to be measured is, for example, produced in a system for producing ultrapure water that includes a pretreatment apparatus, a primary pure water apparatus, and a secondary pure water apparatus. FIG. 5 schematically illustrates a system 100 for producing ultrapure water that produces ultrapure water to be measured by the particle measurement systems of the embodiments described above. The system 100 for producing ultrapure water includes a pretreatment apparatus 110, a primary pure water apparatus 120, and a secondary pure water apparatus 130 in this order.

The secondary pure water apparatus 130 includes a primary pure water tank 131, a pump 132, a heat exchanger 133, a low pressure ultraviolet oxidizer (UV oxidizer) 134, a non-regenerative ion exchanger 135, and an ultrafiltration apparatus 136 in this order. The ultrafiltration apparatus 136 is connected to a point of use (POU) 140 of ultrapure water, and permeate water of the ultrafiltration apparatus 136 is sent to and used in the point of use 140.

The particle measurement systems 1, 2, and 3 of the above-mentioned embodiments are connected to, for example, immediately before the point of use (POU) 140.

The concentration of particles in the ultrapure water produced in the secondary pure water apparatus 130 in the above-mentioned embodiments, that is, for example, the number of particles with a particle diameter of 50 nm or more is, 50 particles/L or more, or 10 particles/L or more. The concentration of particles in the ultrapure water is preferably 1000 particles/L or less to maintain highly dispersed state of particles on the ultrafiltration membrane surface of the particle measurement systems 1, 2, and 3. Such low concentration particles can be accurately measured by using the particle measurement systems 1, 2, and 3 of the embodiments.

### EXAMPLES

A description will now be given of examples. The present disclosure is not limited to the examples described below.

### Example 1

The number of particles in sample water was measured using a particle measurement system same as the system illustrated in FIG. 1. The sample water was prepared by dispersing standard particles made of polystyrene (3000 Series Nanosphere Size Standards manufactured by Thermo Fisher Scientific) with a particle diameter of 50 nm in the ultrapure water produced in the secondary pure water apparatus so that the concentration became 10 particles/L. To the ultrafiltration apparatus 12, 200 ml of the sample water was supplied. In that case, the valve V1 was closed and the valves V2 and V3 were opened to allow permeate water to flow out (accumulation process). Then, the valve V1 was opened while the valves V2 and V3 were closed, and 2 mL of the sample water was supplied to the ultrafiltration apparatus 12. Thus, the particles accumulated in the ultrafiltration apparatus 12 during the accumulation process were allowed to flow into the particle counter (extrusion process). The number of particles (particle concentration) in the water was calculated using the above-described expression (1) from the value measured by the particle counter.

The specifications of the ultrafiltration apparatus and the particle counter used in Example 1 were as follows:
Ultrafiltration apparatus: ultrafiltration apparatus including an ultrafiltration membrane (MIDIKROS manufactured by Repligen, made of modified polyethersulfone (MPES), hollow fiber membrane) with a membrane area of 115 cm² and a nominal pore diameter of 5 kDa; and
Particle counter: Ultra DI 50 Liquid Optical Particle Counter (UDI50) manufactured by PMS.

### Comparative Example 1

Sample water same as that in Example 1 was directly measured with the above-described particle counter (UDI50).

### Comparative Example 2

The ultrafiltration membrane in Example 1 was replaced with a microfiltration membrane shown below, and sample water same as that in Example 1 was used to measure the number of particles in the sample water in the same manner as in Example

1. The number of particles in the water was calculated using the above-described expression (1) from the value measured by the particle counter.

Microfiltration apparatus: microfiltration apparatus including a microfiltration membrane (Microza MF manufactured by Asahi Kasei Corp., made of polyethylene, hollow fiber membrane) with a membrane area of 150 cm² and a nominal pore diameter of 0.1 μm

### Comparative Example 3

In Comparative Example 3, the number of particles was measured by supplying concentrate water directly to the particle counter when cross-flow filtration was performed so that the flow rate of the concentrate water accounted for 90% of the supply flow rate in the ultrafiltration apparatus.

With use of an ultrafiltration apparatus same as that in Example 1, cross-flow filtration was performed by opening the valves V1 and V3 and closing the valve V2 so that the flow rate of permeate water was 1 mL (the flow rate of concentrate water passing through the valve V1 accounted for 90% of the supply flow rate from the supply pipe 10). The number of particles in the concentrate water flowing out of the ultrafiltration apparatus 12 was continuously measured by the particle counter. The particle concentration in the water was calculated from the value measured by the particle counter using a concentration factor.

The results of the number of particles (particles/L) measured in Example 1 to Comparative Example 3 are shown in Table 1. In Table 1 below, the “measured values” indicate values of the number of particles calculated by the above-described expression (1) based on the results of measurement with the particle counter in Example 1, Comparative Example 2, and Comparative Example 3. The number of particles in the sample water is shown in parentheses as a value (particles/L) calculated from amounts of ultrapure water and standard particles.

TABLE 1

| | Number of Particles |
|---|---|
| | Value Converted to |
| | Concentration on Water |
| | To Be Measured |
| Sample Water | (10) |
| Example 1 | 9.91 |
| Comparative Example 1 | 0 |
| Comparative Example 2 | 2.95 |
| Comparative Example 3 | 5.8 |

Table 1 indicates that the particle measurement system of Example 1 could accurately measure 10 particles/L of particles. In Comparative Example 1, low concentration particles could not be measured because the accumulation process was not performed. In Comparative Example 2, the microfiltration membrane was used instead of the ultrafiltration membrane, which resulted in insufficient accumulation of particles. Therefore, the measurement was less accurate than in Example 1. It is also clear that in Example 1, a measured value closest to the concentration of the sample water was obtained. This means that the particles in the sample water could be analyzed as they were because particles did not form “lumps” and therefore the number of particles and the particle diameter were unchanged.

Next, the relationship between a flux of permeate water in the ultrafiltration apparatus and a recovery rate of particles in the accumulation process was examined.

### Example 2

The number of particles in sample water was measured using a particle measurement system same as the system illustrated in FIG. 1. Sample water same as that in Example 1 was supplied to the ultrafiltration apparatus 12. In that case, the valve V1 was closed and the valves V2 and V3 were opened to perform dead-end filtration in the ultrafiltration apparatus 12 and the permeate water was allowed to flow out (accumulation process). In the accumulation process, the filtration time was 15 minutes and the inlet pressure of the ultrafiltration apparatus 12 was 0.16 MPa. The flow rate of permeate water was 300 mL/min and the flux of the permeate water was 27 mm/min in the accumulation process. The valve V1 was opened and the valves V2 and V3 were closed, and the sample water was supplied to the ultrafiltration apparatus 12 so that the flow rate of concentrate water was 2 mL/min. Thus, the particles accumulated in the ultrafiltration apparatus 12 during the accumulation process were allowed to flow into the particle counter (extrusion process). From the particle concentration at the outlet side of the particle counter at that time and the particle concentration in the sample water, the recovery rate of particles from the ultrafiltration apparatus was calculated. The results are shown in Table 2. A higher particle recovery rate indicates a smaller number of particles remaining in the ultrafiltration apparatus after the extrusion process. The accumulation process was also performed under the same conditions, and then the number of particles on the membrane surface was measured by a direct microscopic count method. The results are shown in Table 1.

### Example 3 to Comparative Example 4

The recovery rate of particles and the number of particles on the membrane surface after the accumulation process were calculated in the same manner as in Example 2, except that the flux of permeate water in the accumulation process in Example 2 was changed. The results are shown in Table 2. The flux of permeate water was 40 mm/min in Example 3, 54 mm/min in Example 4, and 90 mm/min in Comparative Example 4.

TABLE 2

| | Filtration | | Number of |
|---|---|---|---|
| | process | | Particles on |
| | (Accumulation | Recovery | Membrane Surface |
| | Process) | Rate (%) | (Number/cm²) |
| Example 2 | Dead-End | 98 | 4 × 10⁷ |
| | Filtration | | |
| Example 3 | Dead-End | 77 | 3.8 × 10¹⁰ |
| | Filtration | | |
| Example 4 | Dead-End | 72 | 5 × 10¹⁰ |
| | Filtration | | |
| Comparative | Dead-End | 58 | 1 × 10¹¹ |
| Example 4 | Filtration | | |

Table 2 shows that, as the number of particles on the membrane increases in the accumulation process, the recovery rate of particles decreases, that is, the number of particles remaining in the ultrafiltration apparatus increases in the extrusion process. In Example 2 to Example 4, particles adhered to the membrane without forming “lumps” so that they were accumulated in the accumulation process. On the other hand, in Comparative Example 4, particles on the membrane surface excessively deposited and thereby formed “lumps”. This is considered to be because a flux of permeate water in the accumulation process is high, causing particles to deposit on the membrane surface and form “lumps” or the like. As a result, the number of particles on the membrane surface increases, and the particles become less likely to detach from the membrane surface, or enter pores of the membrane, thereby being inhibited from flowing out in the extrusion process.

1. A particle measurement method using a particle measurement apparatus, the method comprising:
an accumulation process of filtering ultrapure water to be measured through an ultrafiltration membrane to accumulate particles on the ultrafiltration membrane; and
an extrusion process of forcing the particles accumulated on the ultrafiltration membrane, together with concentrate water by the ultrafiltration membrane, to the particle measurement apparatus.

2. The particle measurement method according to claim 1, wherein, in the accumulation process, dead-end filtration is performed.

3. The particle measurement method according to claim 1, wherein a number of particles on the ultrafiltration membrane after the accumulation process is 4×10⁶ particles/cm² or more and 5×10¹⁰ particles/cm² or less.

4. The particle measurement method according to claim 1, wherein a total amount of concentrate water flowing out of the ultrafiltration membrane in the extrusion process is 0.05 times or more and 0.2 times or less a total amount of the ultrapure water supplied to the ultrafiltration membrane in the accumulation process.

5. The particle measurement method according to claim 1, wherein continuous measurement is performed by alternately performing the accumulation process and the extrusion process.

6. The particle measurement method according to claim 1, wherein an entire amount of the ultrapure water supplied to the ultrafiltration membrane in the extrusion process is allowed to flow out as the concentrate water.

7. The particle measurement method according to claim 1, wherein a flow rate of the concentrate water by the ultrafiltration membrane in the extrusion process is 90% or more of a supply flow rate of the ultrapure water to the ultrafiltration membrane.

8. The particle measurement method according to claim 1, wherein a number of particles in the ultrapure water is 1 particle/L or more and 1000 particles/L or less.

9. A particle measurement system comprising:
an ultrafiltration apparatus;
a particle measurement apparatus connected to a concentrate side of the ultrafiltration apparatus;
an accumulation mechanism configured to filter ultrapure water by supplying the ultrapure water to the ultrafiltration apparatus to accumulate particles in the ultrafiltration apparatus; and
an extrusion mechanism configured to force the particles accumulated in the ultrafiltration apparatus to the particle measurement apparatus together with concentrate water in the ultrafiltration apparatus.

10. The particle measurement system according to claim 9, wherein a number of particles accumulated on a membrane surface of the ultrafiltration membrane by the accumulation mechanism is 4×10⁶ particles/cm² or more and 5×10¹⁰ particles/cm² or less.

11. The particle measurement system according to claim 9, wherein the particle measurement apparatus measures particles online.

12. The particle measurement system according to claim 9, wherein the accumulation mechanism and the extrusion mechanism are alternately operated.

13. The particle measurement system according to claim 9, comprising:
a first valve provided on a concentrate side of the ultrafiltration apparatus;
a second valve provided on a permeate side of the ultrafiltration apparatus; and
a control apparatus configured to automatically control the first valve and the second valve, wherein:
the control apparatus constitutes the accumulation mechanism by reducing an opening degree of the first valve and opening the second valve; and
the control apparatus constitutes the extrusion mechanism by opening the first valve and the second valve and reducing the opening degree of the second valve so that a flow rate at the first valve is 90% or more of a supply flow rate to the ultrafiltration apparatus.

14. The particle measurement system according to claim 13, wherein the control apparatus constitutes the accumulation mechanism by closing the first valve and opening the second valve, and constitutes the extrusion mechanism by opening the first valve and closing the second valve.
